(19) United States
(12) Patent Application Publication
(10) Pub. No.: US 2014/0053307 A1
(43) Pub. Date: Feb. 27, 2014

(54) PROTECTING MASK

(71) Applicant: Han-Yuan Cheng, Kaohsiung City (TW)

(72) Inventor: Han-Yuan Cheng, Kaohsiung City (TW)

(73) Assignee: POLISON CORPORATION, Kaohsiung City (TW)

(21) Appl. No.: 13/595,079

(22) Filed: Aug. 27, 2012

(51) Int. Cl. A61F 9/06

(52) U.S. Cl. 2/8.2

(57) ABSTRACT

A protecting mask includes a main body, a mask unit, and multiple connecting members. The main body includes a connecting wall having an inner surface and an outer surface and formed with multiple first through holes. The mask unit is disposed at the outer surface of the connecting wall and is formed with multiple second through holes corresponding to the first through holes, respectively. Each connecting member includes an engaging portion removably engaging the mask unit, and a resilient protruding portion extending from the engaging portion through a corresponding pair of the first and second through holes, and engaging the inner surface of the connecting wall.

### BACKGROUND OF THE INVENTION

1. Field of the Invention

This invention relates to a protecting mask, more particularly to a protecting mask for protecting a face of a wearer during a welding operation.

2. Description of the Related Art

During a welding operation, a chemical operation, etc., workers and operators have to wear a mask or a hamlet for protecting their faces from welding sparks or splash of chemicals.

FIGS. 1 and 2 show a conventional protecting mask 1 disclosed in Taiwanese Patent No. 558957. The conventional protecting mask 1 includes a head wearing unit 11, a main body 12, a mask unit 13, a plurality of engaging members 14, and a plurality of locking components 15. The main body 12 includes a connecting wall 121, and a positioning element 122 disposed at an outer surface of the connecting wall 121. The mask unit 13 is formed with a positioning hole 130 having a first part 131 and a second part 132 that is smaller than the first part 131.

When assembling the conventional protecting mask 1, the mask unit 13 is attached to the outer surface of the connecting wall 121 and the positioning element 122 extends through the first part 131 of the positioning hole 130. Then, the mask unit 13 is moved from a position indicated by dashed lines to a position indicated by solid lines along a direction indicated by an Arrow (A) in FIG. 2. In order to secure the mask unit 13 to the main body 12, the engaging members 14 are respectively disposed at openings of a plurality of elongated through holes 133 that are formed in the mask unit 13, and are connected to the connecting wall 121 of the main body 12 by the locking components 15 (e.g., screws, rivets, etc.), respectively. Afterward, the engaging members 14 are rotated with respect to the mask unit 13 in a direction (B), so that the engaging members 14 are misaligned respectively with the elongated through holes 133 and engage and abut against the mask unit 13. As a result, the mask unit 13 is assembled to the main body 12.

However, since the engaging members 14 are connected to the main body 12 by the locking components 15, an additional tool is required during assembly of the conventional protecting mask 1. In addition, since the size of locking components 15 is relatively small, attention has to be paid to the locking components 15 when assembling the engaging members 14 with the main body 12. Accordingly, manufacturing efficiency of the conventional protecting mask 1 is relatively low. Further, the locking components 15 also increase the manufacturing cost of the conventional protecting mask 1.

Moreover, the locking components 15 are generally made of metal, which may conduct the heat of the welding sparks, as well as electric current, injuring the worker/operator wearing the conventional protecting mask 1 during a welding operation.

### SUMMARY OF THE INVENTION

Therefore, the object of this invention is to provide a protecting mask that can overcome the aforesaid drawbacks of the prior art.

According to this invention, a protecting mask includes a main body, a mask unit, and a plurality of connecting members. The main body includes a connecting wall that has an inner surface and an outer surface and that is formed with a plurality of first through holes. The mask unit is disposed at the outer surface of the connecting wall and is formed with a plurality of second through holes that correspond to the first through holes, respectively. The connecting members are made of an insulating material, and each includes an engaging portion that removably engages the mask unit, and a resilient protruding portion that extends from the engaging portion. The resilient protruding portion includes an extending segment connected to the engaging portion and extending through a corresponding pair of the first and second through holes, and a locking segment connected to the extending segment and engaging the inner surface of the connecting wall.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is an exploded perspective view of a conventional protecting mask disclosed in Taiwanese Patent No. 558957;

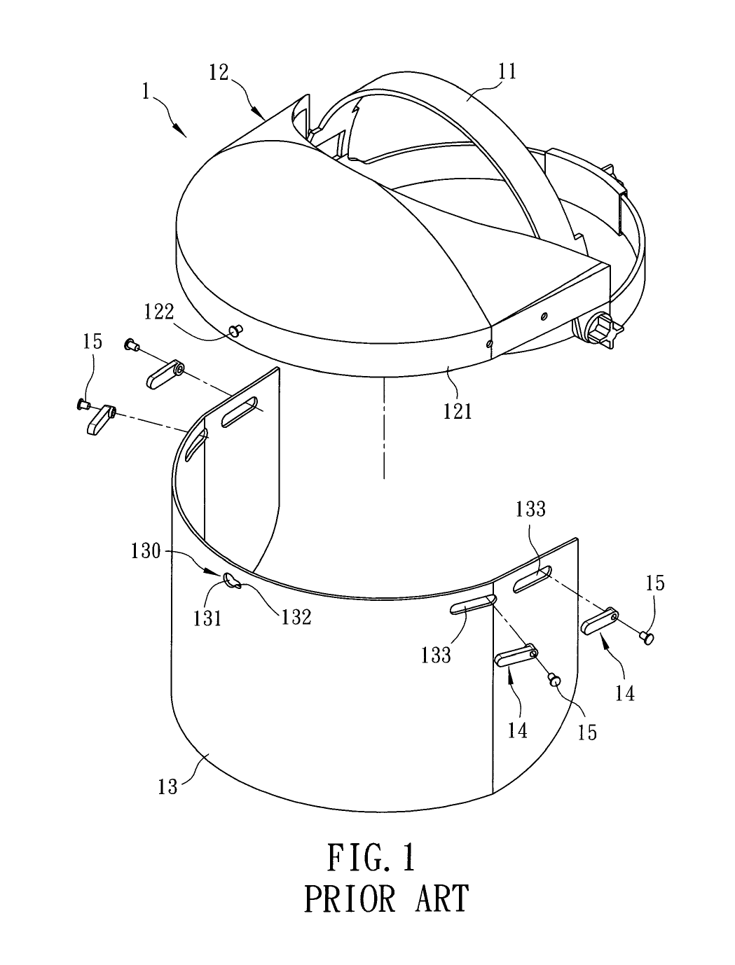

FIG. 2 is a perspective view of the conventional protecting mask that has been assembled;

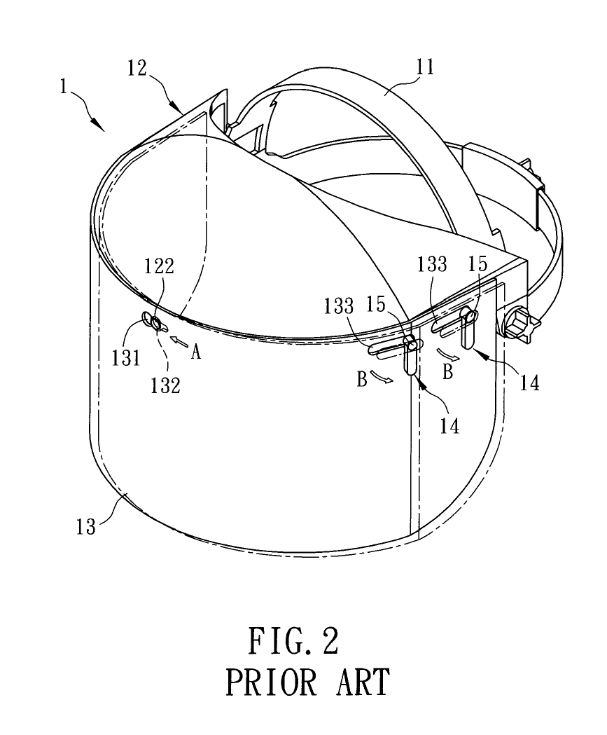

FIG. 3 is an exploded perspective view of a first preferred embodiment of a protecting mask according to this invention;

FIG. 4 is a partly sectional view illustrating assembly relationships among a connecting member, a first through hole, and a mask unit of the first preferred embodiment;

FIG. 5 is a perspective view of the protecting mask that has been assembled;

FIG. 6 is a side view of modification of the connecting member of the first preferred embodiment; and

FIG. 7 is an exploded perspective view of a second preferred embodiment of a protecting mask according to this invention.

### DETAILED DESCRIPTION OF THE PREFERRED EMBODIMENTS

Before the present invention is described in greater detail with reference to the preferred embodiments, it should be noted that the same reference numerals are used to denote the same elements throughout the following description.

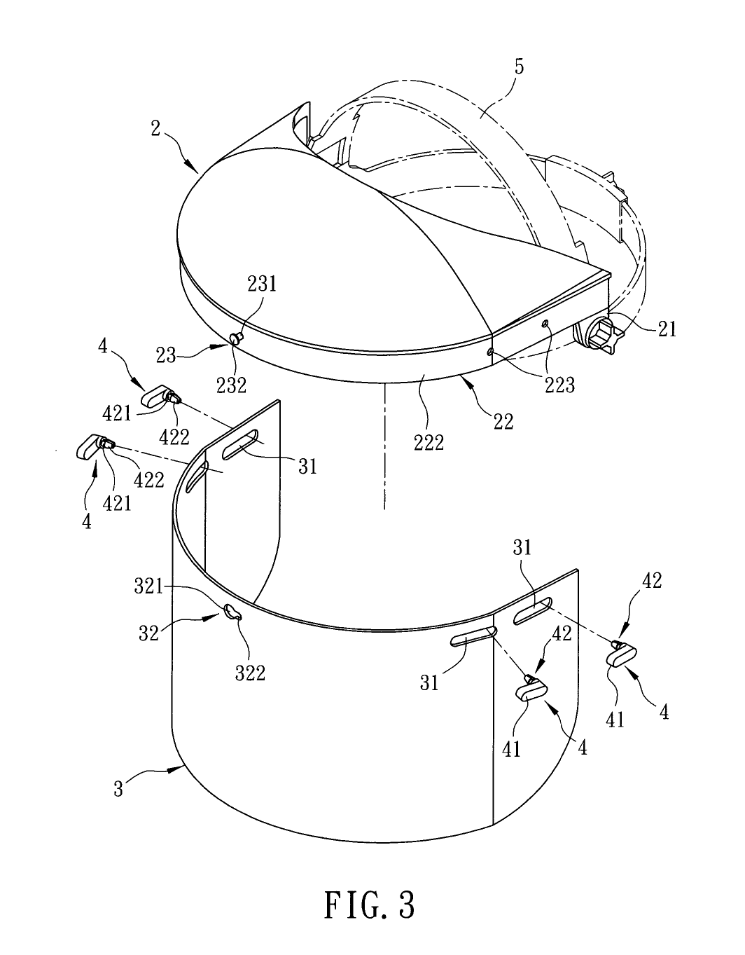

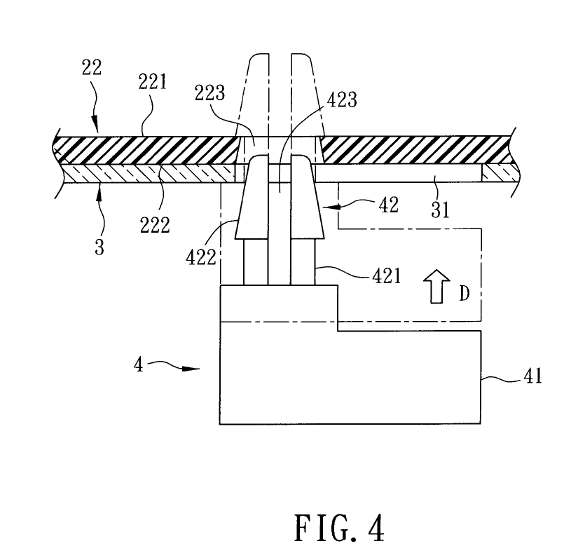

FIGS. 3 and 4 shows a first preferred embodiment of a protecting mask according to this invention. The protecting mask is adapted to be connected to a head wearing unit 5, and includes a main body 2, a mask unit 3, and a plurality of connecting members 4.

The main body 2 includes a connecting wall 22 and a positioning element 23. The connecting wall 22 has an opposite pair of rear ends 21 adapted to be pivotally connected to the head wearing unit 5. The connecting wall 22 further has an inner surface 221 and an outer surface 222 and is formed with a plurality of first through holes 223. Each of the first through holes 223 is in the shape of a frustum, and has an inner diameter tapering from the outer surface 222 of the connecting wall 22 to the inner surface 221 of the connecting wall 22. The positioning element 23 protrudes outwardly from the outer surface 222 of the connecting wall 22, and includes a neck portion 231 and a head portion 232 that has a cross-sectional area greater than a cross-sectional area of the neck portion 231.

The mask unit 3 is disposed at the outer surface 222 of the connecting wall 22, and is formed with a plurality of second through holes 31, each of which is in an elongated shape and corresponds to a respective one of the first through holes 223. The mask unit 3 is further formed with a positioning hole 32, and the positioning element 23 of the connecting wall 22 removably engages the positioning hole 32. The positioning hole 32 has a first part 321 that allows the head portion 232 of the positioning element 23 to extend therethrough, and a second part 322 that has an area smaller than an area of the first part 321, that is in spatial communication with the first part 321, and that allows the neck portion 231 of the positioning element 23 to extend therethrough.

Each of the connecting members 4 is integrally made of an insulating material, and includes an engaging portion 41 that is an elongated block, and a resilient protruding portion 42 that extends from the engaging portion 41. The engaging portion 41 is rotatable with respect to the mask unit 13, and removably engages and abuts against the mask unit 13. The resilient protruding portion 42 includes an extending segment 421 connected to the engaging portion 41 and extending through a corresponding pair of the first and second through holes 223, 31, and a locking segment 422 connected to the extending segment 421 and engaging the inner surface 221 of the connecting wall 22. In particular, the locking segment 422 of the resilient protruding portion 42 is in the shape of a cone. In this embodiment, the resilient protruding portion 42 is formed with a gap 423 extending along the extending segment 421 and the locking segment 422.

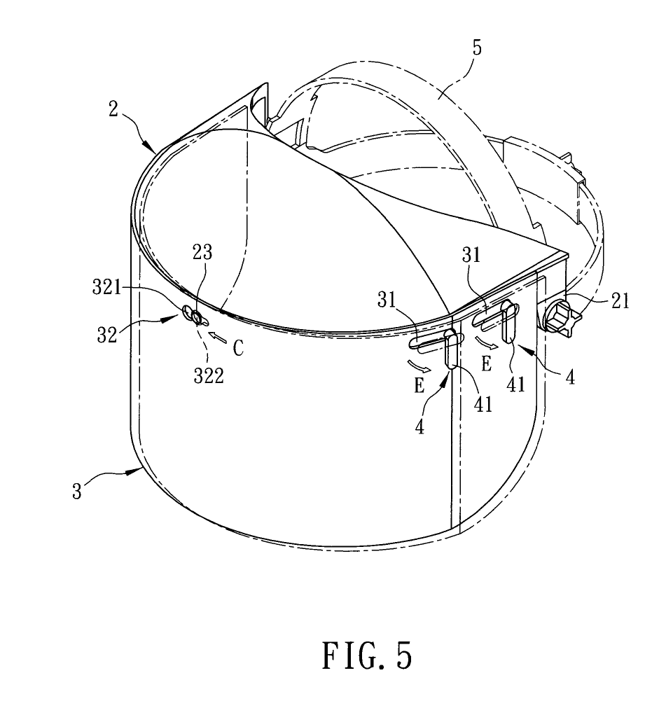

Referring to FIGS. 3, 4 and 5, the positioning element 23 is first extended through the first part 321 of the positioning hole 32 of the mask unit 3 during assembly of the protecting mask. Then, the mask unit 3 is moved from a position indicated by dashed lines to a position indicated by solid lines along a direction (C) in FIG. 5, so that the neck portion 231 of the positioning element 23 slides into and engages the second part 322 of the positioning hole 32. At this time, by virtue of the head portion 232 of the positioning element 23 engaging the mask unit 3 at the second part 322 of the positioning hole 32, the mask unit 3 preliminarily engages the connecting wall 22 of the main body 2, and the first through holes 223 of the connecting wall 22 are in spatial communication with the second through holes 31 of the mask unit 3, respectively. Then, the resilient protruding portion 42 of each of the connecting members 4 is inserted through said the corresponding pair of the first and second through holes 223, 31 in a direction (D) shown in FIG. 4. In particular, the gap 423 in the protruding portion 42 allows the locking segment 422 to be compressed inwardly so that a width of the locking segment 422 is reduced so as to smoothly slide through the first through hole 223 in the corresponding pair of the first and second through holes 223, 31. After sliding through the first through hole 223, the locking segment 422 is restored to the original width thereof and abuts against the inner surface 221 of the connecting wall 22 as shown by dashed lines in FIG. 4. At this time, the connecting member 4 is at a releasing position, where the engaging portion 41 is aligned with one of the second through holes 31 that corresponds to the resilient protruding portion 42.

Then, the engaging portion 41 is moved in a direction (E) shown in FIG. 5 to result in pivot action of the connecting member 4 relative to the mask unit 3, until the connecting member 4 is at an engaging position, where the engaging portion 41 is misaligned with said one of the second through holes 31 and engages and abuts against the mask unit 3, such that the mask unit 3 and the main body 2 are fully assembled together. Finally, the main body 2 is pivotally connected to the head wearing unit 5 at the rear ends 21, and thus the mask unit 3 is pivotable with respect to the head wearing unit 5 together with the main body 2.

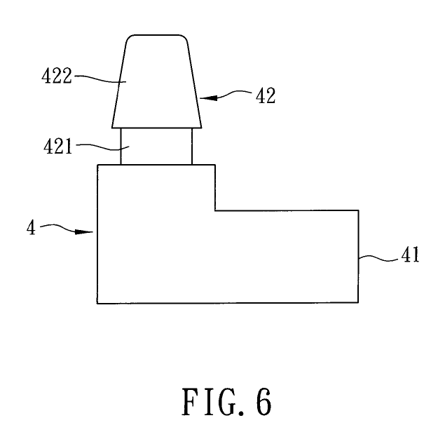

The resilient protruding portion 42 of each of the connecting members 4 is not limited to the disclosure shown in FIG. 4. As shown in FIG. 6, the gap 423 of the resilient protruding portion 42 may be omitted, and the resilient protruding portion 42 may still slide through the first through hole 223 in the corresponding pair of the first and second through holes 223, 31 due to resiliency thereof to abut against the inner surface 221 of the connecting wall 22. Other configurations of the resilient protruding portion 42, which can smoothly slide through the first through hole 223 and abut against the inner surface 221 of the connecting wall 22, may be used without departing from the scope of this invention.

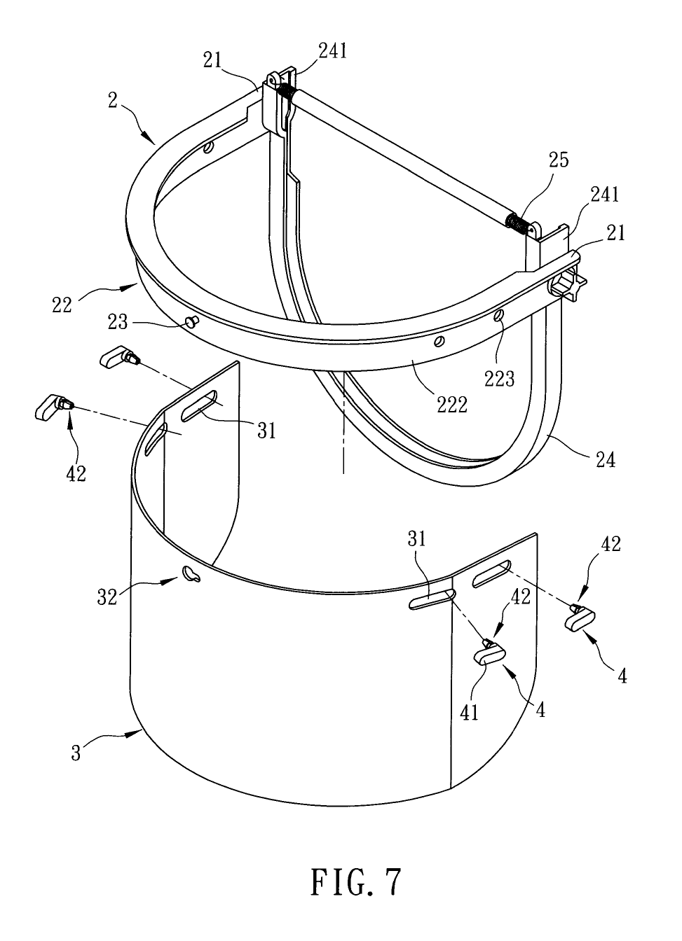

FIG. 7 shows a second preferred embodiment of the protecting mask according to this invention. In the second preferred embodiment, it is not necessary for the main body 2 to be connected with a head wearing unit 5, and the main body 2 further includes an U-shaped lower member 24 that is disposed under the connecting wall 22 and that has an opposite pair of upper ends 241 pivotally and respectively connected to the rear ends 21 of the connecting wall 22. The main body 2 further includes an elastic member 25 disposed between the upper ends 241 of the lower member 24. The connecting wall 22, the lower member 24 and the elastic member 25 cooperatively define a receiving space for receiving a head of a user wearing the protecting mask. When wearing the protecting mask of this embodiment, a jaw of the user rests upon the lower member 24 and the elastic member 25 abuts against a rear part of the head of the user so as to ensure that the protecting mask is worn stably.

It should be noted that the elastic member 25 is a spring in the second embodiment, and the lower member 24 is made of a rigid material. Configurations of the elastic member 25 and the lower member 24 are not limited to the disclosure of this embodiment, and may be modified and varied in other embodiments. For example, the lower member 24 may be a strip made of a soft material or an elastic band.

To sum up, compared with the conventional protecting mask 1 disclosed in Taiwanese Patent No. 558957, since the engaging portion 41 and the resilient protruding portion 42 of each of the connecting members 4 are integrally made of an insulating material that is cheaper than metal, the protecting mask of this invention does not need the locking components 15, the number of components and the manufacturing cost of the protecting mask are both reduced, and assembly of the protecting mask is relatively easy and efficient according to this invention. Further, since the connecting members 4 are made of an insulating material, the connecting members 4 will not conduct heat of welding sparks and electric current so that the user wearing the protecting mask of this invention is free from injury attributed to the heat of welding sparks and electric current. In addition, by virtue of the frustum shape of the first through holes 223, engagement between the locking segment 422 of the resilient protruding portion 42 of each of the connecting members 4 and the inner surface 221 of the connecting wall 22 is relatively stable.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. A protecting mask, comprising:
a main body including a connecting wall that has an inner surface and an outer surface and that is formed with a plurality of first through holes;
a mask unit disposed at said outer surface of said connecting wall and formed with a plurality of second through holes that correspond to said first through holes, respectively; and
a plurality of connecting members made of an insulating material, each of said connecting members including
an engaging portion that removably engages said mask unit, and
a resilient protruding portion that extends from said engaging portion and that includes an extending segment connected to said engaging portion and extending through a corresponding pair of said first and second through holes, and a locking segment connected to said extending segment and removably engaging said inner surface of said connecting wall.

2. The protecting mask as claimed in claim 1, wherein said protruding portion of each of said connecting members is formed with a gap extending along said extending segment and said locking segment.

3. The protecting mask as claimed in claim 1, wherein each of said first through holes has an inner diameter tapering from said outer surface of said connecting wall to said inner surface of said connecting wall.

4. The protecting mask as claimed in claim 1, wherein said locking segment is in the shape of a cone.

5. The protecting mask as claimed in claim 1, wherein each of said second through holes has an elongated shape, and said engaging portion of each of said connecting members is an elongated block.

6. The protecting mask as claimed in claim 1, wherein said mask unit is further formed with a positioning hole, and said main body further includes a positioning element protruding outwardly from said outer surface of said connecting wall and removably engaging said positioning hole.

7. The protecting mask as claimed in claim 6, wherein:
said positioning element includes a neck portion and a head portion that has a cross-sectional area greater than a cross-sectional area of said neck portion; and
said positioning hole has a first part that allows said head portion of said positioning element to extend therethrough, and a second part that has an area smaller than an area of said first part, that is in spatial communication with said first part, and that allows said neck portion of said positioning element to extend therethrough.

8. The protecting mask as claimed in claim 1, wherein:
said connecting wall is curved, and further has an opposite pair of rear ends;
said main body further includes a lower member that is disposed under said connecting wall and that has an opposite pair of upper ends pivotally and respectively connected to said rear ends of said connecting wall, and an elastic member disposed between said upper ends of said lower member;
said connecting wall, said lower member and said elastic member cooperatively define a receiving space for receiving a head of a user wearing said protecting mask.

9. The protecting mask as claimed in claim 8, wherein said elastic member is a spring.

10. The protecting mask as claimed in claim 1, wherein said engaging portion of each of said connecting members is pivotable with respect to said mask unit between an engaging position, where said engaging portion is misaligned with one of said second through holes that corresponds to said resilient protruding portion and engages and abuts against said mask unit, and a releasing position, where said engaging portion is aligned with said one of said second through holes.
